(19) United States
(12) Patent Application Publication
(10) Pub. No.: US 2004/0109851 A1
(43) Pub. Date: Jun. 10, 2004

(54) Use of immature dendritic cells to silence antigen specific cd8cell function

(76) Inventors: Madhav V. Dhodapkar, New York, NY (US); RalphM M Stein, Westport, CT (US); Nina Bhardwaj, West Orange, NJ (US)

Correspondence Address: MORGAN & FINNEGAN, L.L.P., 345 PARK AVENUE, NEW YORK, NY, 10154, US

(21) Appl. No.: 10/451,039

(22) PCT Filed: Dec. 21, 2001
(86) PCT No.: PCT/US01/50578

(30) Foreign Application Priority Data
Dec. 22, 2000 (US) 60257998

(51) Int. Cl. A61K 48/00; A61K 39/395; C12N 5/08

(52) U.S. Cl. 424/93.21; 424/163.1; 424/130.1; 424/93.7; 435/372

(57) ABSTRACT

This invention provides methods for silencing a pre-existing immune response in a mammal, as for example, in the setting of autoimmune diseases. The method comprises administering to a mammal immature dendritic cells which have been contacted in vitro with an antigen, or to target the antigen to immature dendritic cells in vivo, in order to silence and/or suppress a pre-existing CD8+T cell immune response and induce IL-10 producing CD8+T cells in said mammal. This invention further relates to methods for propagating immature dendritic cells, for maintaining immaturity by modification ex vivo, and uses thereof, including generation of regulatory T cells for passive immunotherapy. The present invention also relates to compositions and kits comprising immature dendritic cells and antigens.

### STATEMENT OF PRIORITY

This application claims priority to U.S. Provisional Application No. 60/257,998, filed Dec. 22, 2000, the entire contents of which is herein incorporated by reference.

### STATEMENT OF GOVERNMENT SUPPORT

This invention was supported in part by an investigator award from the Cancer Research Institute (to MVD) and grants from the National Institutes of Health (CA 81138 (to MVD); and MO-RR00102 to the Rockefeller GCRC), Al 40045 to RMS, and Al to NB. The Government of the United States of America has certain rights in this invention.

### TECHNICAL FIELD OF THE INVENTION

This invention relates to methods for silencing and/or suppressing a pre-existing immune response in a mammal. This invention further relates to methods for propagating immature dendritic cells, and uses thereof. In particular, this invention relates to the use of immature dendritic cells for silencing and/or suppressing pre-existing antigen specific CD8+T cell function in a mammal. The present invention also relates to compositions and kits comprising immature dendritic cells and antigens.

### BACKGROUND OF THE INVENTION

Dendritic cells are specialized antigen presenting cells which are critical for eliciting T cell mediated immune responses (Steinman, 1991; Caux et al. 1995b; Hart and McKenzie, 1990; Austyn, 1987). Dendritic cells activate both CD4⁺ helper T cells and CD8+ cytotoxic T cells in vivo (Inaba et al. 1990a; Inaba et al. 1990b; Porgador and Gilboa, 1995). Dendritic cells typically reside in nonlymphoid tissue in an immature form where they are capable of internalizing antigens. After antigen uptake, dendritic cells migrate from nonlymphoid tissues to regional lymph nodes as an important step in the generation of T cell-mediated immune responses.

Inflammatory stimuli switch dendritic cells to an immunostimulatory mode. This process is termed “maturation” and is associated with changes in dendritic cell phenotype and function, including up regulation of co-stimulatory and adhesion molecules and expression of distinct chemokine receptors (Cella et al. 1997).

Depending on their maturational state, dendritic cells may perform different functions in the immune system. For example, due to their potency as antigen presenting cells, there has been considerable interest in utilizing dendritic cells as adjuvants to enhance immunity against cancer and viral infection. Recent reports indicate that induction of immunity requires mature dendritic cells (Inaba et al. 2000; Dhodapkar et al. 2000; Labeur et al. 1999), whereas immature dendritic cells have been reported to result in no induction of immunity or poor clinical outcomes in the context of cancer or viral infections (Panelli et al. 2000).

At present, few studies have been done on the immunosuppressive properties of immature dendritic cells. U.S. Pat. No. 5,871,728 reports methods for using immature dendritic cells to enhance tolerogenicity in a mammal to a transplanted graft from a donor mammal. Similarly, Lutz et al. 2000 reports that immature dendritic cells may play a role in prolonging allograft survival. However, both of these studies report that the immature dendritic cells must be administered in advance of transplantation, before an immune response has been mounted, to induce tolerance. Therefore, they do not address the problems present in autoimmune diseases where an autoreactive T cell response already exists. Examples of such diseases include, juvenile diabetes, multiple sclerosis, psoriasis, systemic lupus erythematosus (SLE), and rheumatoid arthritis.

Repetitive stimulation with immature dendritic cells has recently been reported to induce IL-10 producing regulatory CD4⁺helper T cells in vitro (Jonuleit et al. 2000). However, this study does not disclose or suggest methods for suppression of a pre-existing cytotoxic CD8+T cell response in vivo.

### SUMMARY OF THE INVENTION

This invention provides a method for silencing and/or suppressing a pre-existing antigen-specific T cell immune response in a mammal which is characterized by the presence of antigen specific CD8+T cells. The method comprises administering to an individual in need of treatment immature dendritic cells, which have been contacted with an antigen of interest, in an amount sufficient to suppress or inhibit the function of antigen specific CD8+T cells in vivo.

In one embodiment of the invention the tissue source for dendritic cells is blood or bone marrow. A preferred tissue source is blood, and more preferably human blood.

In another embodiment, this invention relates to a composition comprising immature dendritic cells which is suitable for inhibiting antigen specific CD8+T-cell function.

In a further embodiment, this invention relates to a kit for inhibiting antigen specific CD8+T-cell function. The kit comprises immature dendritic cells and antigen, or immature dendritic cells which have already been contacted with antigen.

In a further embodiment, the present invention also relates to in vivo targeting of immature dendritic cells which are resident in tissues or those which are elicited after contact with cytokines such as G-CSF or FLT-3 ligand, for inhibiting the function of CD8+T cells in vivo.

This invention also provides methods for generating immature dendritic cells that include modifications such as treatment or expression of cytokines which inhibit maturation of dendritic cells and maintain dendritic cells in an immature state in vivo (for example, by transforming the immature dendritic cells with at least one vector comprising a gene encoding TGF-β and/or IL-10 family proteins, preferably IL-10) and which may be used to prepare therapeutic compositions.

This invention further provides methods for isolating and administering the immature dendritic cells of the invention.

This invention also provides methods for stimulating production of regulatory T cells either in vitro for passive immunotherapy or in vivo for active immunization, in order to dampen or inhibit pre-existing antigen specific T cell function. This invention also provides methods for measuring or monitoring the regulatory T cells.

This invention further provides a method of treating autoimmune diseases, for example, juvenile diabetes, multiple sclerosis, psoriasis, systemic lupus erythematosus (SLE), rheumatoid arthritis, with a therapeutically effective amount of immature dendritic cells to induce silencing or suppression of pre-existing self or autoreactive T cells.

This invention further provides a method of treating a transplant recipient with a therapeutically effective amount of immature dendritic cells to induce silencing or suppression of T cells which are specific for the transplanted organ or other foreign transplanted antigens. This strategy may be effective for the therapy or prevention of graft versus host disease after bone marrow/stem cell transplantation or therapy of graft rejection in solid organ transplantation.

### FIGURE LEGENDS

FIGS. 1A through 1F. Immune responses in uncultured T cells. FIGS. 1A and 1B: MP, gag and influenza specific IFN-γ producing cells from pre and post DC immunization were quantified in freshly isolated uncultured PBMCs using an ELISPOT assay. Data for influenza specific cells is per 10⁵ cells. SEM for all measurements is <20%. SFC=spot forming cells. FIGS. 1C and 1D: Pre and post immunization samples were thawed together and assayed for antigen specific T cells secreting IFN-γ, IL-4 and IL-10 using a 16 hour ELISPOT assay. Antigens were HLA A2.1 restricted peptides from influenza matrix (MP), HIV-gag (gag) and CMVpp65 (CMV). Positive controls for the assays included SEA for IFN-γ and IL-10 and PHA for IL-4 (not shown). SEM for all measurements is <20%. FIG. 1E: Use of peptide pulsed DCs as APCs in the ELISPOT assay. Pre and post immunization specimens were examined using peptide pulsed mature DCs as APCs (PBMC: DC ratio 30:1) in the ELISPOT. SEM for all measurements is <20%. FIG. 1F: Quantification of MP specific T cells using MHC tetramers in uncultured cells. Pre/post immunization specimens were stained with A*0201-MP tetramers at 37° C. and analyzed by flow cytometry. Data shown are gated for CD8+T cells and expressed as percent CD8+T cells binding A*0201-MP tetramer.

FIGS. 2A through 2C. T cell recall assays in culture post DC immunization. Pre and post immunization specimens were thawed and co-cultured with MP pulsed DCs (unpulsed DCs as controls) for 7 days. After 7 day culture, MP specific T cells were quantified by MHC tetramers (FIG. 2A); ELISPOT (FIG. 2B) and CTL assay (FIG. 2C). FIG. 2A MHC Tetramer assay: Data are expressed as percent CD8+T cells binding A*0201-MP tetramer. FIG. 2B ELISPOT assay: On day 7, cells were restimulated with specific antigen (MP 10 μg/ml) (unrestimulated as controls) and antigen specific interferon-γ producing cells quantified using an ELISPOT. SEM for all measurements is <30%. FIG. 2C CTL assay: MP specific lysis was measured using MP pulsed T2 targets. Data shown are after subtracting lysis with control unpulsed T2 targets and from cells after expansion using unpulsed DCs.

FIGS. 3A through 3B. Priming of KLH specific T cells in vivo. FIG. 3A: Antigen dependent proliferation. Pre and post immunization PBMCs were thawed together and cultured in the absence or presence of KLH (10 μg/ml). Data shown are KLH specific proliferation after subtracting ³H TdR incorporation in control wells. SEM for all measurements is <30%. FIG. 3B: KLH specific IFN-γ and IL-4 producing cells from pre and post DC immunization were quantified in freshly isolated uncultured PBMCs using an ELISPOT assay. KLH specific spot forming cells (SFCs) calculated after subtracting data from control wells without antigen.

FIGS. 4A and 4B. Kinetics of the antigen specific T cell response after the injection of influenza matrix (MP) pulsed immature dendritic cells. MP specific interferon- , IL-10, and IL-4 producing T cells were quantified in uncultured PBMCs using an ELISPOT assay. MP specific lytic effectors were quantified after 7 day culture with MP pulsed DCs. Cytolysis data shown are for MP specific lysis of T2 cells as targets (ET ratio 20:1). 1A: Im2; 1B: Im1

FIGS. 5A and 5B. Suppressor Assays: FIG. 5A. Presence of peptide specific regulatory T cells in blood, 7 days after injection. Pre-immunization or day 7 post immunization blood mononuclear cells (2×10⁵ cells/well for Im1, and 3×10⁵ cells/well for Im2), were cultured overnight, either separately or together, in the presence of mature DCs pulsed with HLA A*0201 restricted peptides from MP, LMP-2, and gag at DC: PBMC ratio of 1:60. Antigen specific interferon- producing cells were quantified by an ELISPOT assay. FIG. 5B. Characterization of peptide specific regulatory T cells. PBMCs (3×10⁵ cells/well) from recovery specimens (d 180) of one of the subjects (Im2) were mixed (ratio 1:1) with day 7 specimens, either unseparated, after CD8+T cell depletion, or cultured as physically separated in transwell cultures, or in the presence of rIL-2 (100 U/ml). Antigen specific interferon- producing cells were quantified by an ELISPOT assay after overnight culture in the presence of DCs pulsed with MP, EBV-LMP2 or HIV-gag, at DC:PBMC ratio of 1:60.

### DETAILED DESCRIPTION OF THE INVENTION

This invention provides a method for silencing and/or suppressing pre-existing antigen specific T cell function in a mammal. The invention is based on the discovery that immature dendritic cells are capable of inhibiting or dampening preexisting antigen specific CD8+T cell function when administered in vivo. The immature dendritic cells of the invention may be produced in amounts suitable for various immunological interventions for the prevention and treatment of disease.

In one embodiment, the method comprises administering to a mammal, immature dendritic cells, which have been contacted in vitro with an antigen, in an amount sufficient to silence and/or suppress a pre-existing CD8+T cell immune response in the mammal.

In another embodiment, immature dendritic cells are administered or mobilized in vivo, for example, by administering FLT-3 ligand which elicits circulating immature dendritic cells, and allowed to contact endogenous antigen in vivo. The immature dendritic cells may also be modified ex vivo, for example, using vectors expressing IL-10, to help keep them in an immature state after administering them to a subject in vivo. Alternatively, contact with antigen, vectors, or other antigen delivery systems, may be enhanced in vivo via specific uptake and entry receptors on the dendritic cells such as DEC-205 (Hawiger et al., 2001), or other methods known to those skilled in the art (Mellman and Steinman, 2001).

Isolation of Immature Dendritic Cells from a Tissue Source

The starting material for isolating immature dendritic cells is a tissue source comprising immature dendritic cells or their progenitors, which are capable of proliferating, preferably, in vitro. Methods for isolating and culturing immature dendritic cells are disclosed in U.S. Pat. No. 5,994,126 and published PCT Application No. WO 97/29182, the entire contents of which are incorporated herein by reference. Briefly, appropriate tissue sources for isolating immature dendritic cells include spleen, afferent lymph, bone marrow, blood, and cord blood, as well as blood cells elicited after administration of cytokines such as G-CSF or FLT-3 ligand.

In one embodiment, a tissue sources may be treated prior to culturing with substances that stimulate hematopoiesis, such as, for example, G-CSF and FLT-3 lingand, in order to increase the proportion of dendritic cell precursors relative to other cell types. Other examples include, but are not limited to, GM-CSF, M-CSF, TGF-Beta, and thrombopoietin. Such pretreatment may also remove cells which may compete with the proliferation of the dendritic cell precursors or inhibit their survival.

Pretreatment may also be used to make the tissue source more suitable for in vitro culture. Those skilled in the art would recognize that the method of treatment will likely depend on the particular tissue source. For example, spleen or bone marrow would first be treated so as to obtain single cells followed by suitable cell separation techniques to separate leukocytes from other cell types as described in U.S. Pat. Nos. 5,851,756 and 5,994,126, which are herein incorporated by reference. Treatment of blood would preferably involve cell separation techniques to separate leukocytes from other cell types including red blood cells (RBCs) which are toxic. Removal of RBCs may be accomplished by standard methods known in the art.

In a preferred embodiment of the invention the tissue source is blood or bone marrow. Blood is the more preferred tissue source, and most preferred is human blood.

In a further embodiment, immature dendritic cells are derived from multipotent blood monocyte precursors (See, WO 97/29182). These multipotent cells typically express CD14, CD32, CD68 and CD115 monocyte markers with little or no expression of CD83, or p55 or accessory molecules such as CD40 and CD86. When cultured in the presence of cytokines such as a combination of GM-CSF and IL-4 or IL-13 as described below, the multipotent cells give rise to the immature dendritic cells. The immature dendritic cells can be modified (for example using vectors expressing IL-10) (Buelens et al. 1997), to keep them in an immature state in vitro or in vivo.

Those skilled in the art would recognize that any number of modifications may be introduced to the disclosed methods for isolating immature dendritic cells and maintaining them in an immature state in vitro and in vivo having regard to the objects of the several embodiments of the invention here disclosed.

Culturing of Immature Dendritic Cells

Cells obtained from the appropriate tissue source are cultured to form a primary culture, preferably, on an appropriate substrate in a culture medium supplemented with granulocyte/macrophage colony-stimulating factor (GM-CSF), a substance which promotes the differentiation of pluripotent cells to immature dendritic cells as described in U.S. Pat. No. 5,851,756, which is herein incorporated by reference, and U.S. Pat. No. 5,994,126. In a preferred embodiment, the substrate would include any tissue compatible surface to which cells may adhere. Preferably, the substrate is commercial plastic treated for use in tissue culture.

To further increase the yield of immature dendritic cells, other factors, in addition to GM-CSF, may be added to the culture medium which block or inhibit proliferation of non-dendritic cell types. Example of factors which inhibit non-dendritic cell proliferation include Interleukin4 (IL-4) and/or Interleukin-13 (IL-13), which are known to inhibit macrophage proliferation. The combination of these substances increases the number of immature dendritic cells present in the culture by preferentially stimulating proliferation of the dendritic cell precursors, while at the same time inhibiting growth of non-dendritic cell types.

Thus, by way of illustration, an enriched population of immature dendritic cells can be generated from blood monocyte precursors, for example, by plating mononuclear cells on plastic tissue culture plates and allowing them to adhere. The plastic adherent cells are then cultured in the presence of GM-CSF and IL-4 in order to expand the population of immature dendritic cells. A combination of GM-CSF and IL-4 at a concentration of each of between about 200 to about 2000 U/ml, more preferably between about 500 and 1000 U/ml, and most preferably about 800 U/ml (GM-CSF) and 1000 U/ml (IL-4) produces significant quantities of the immature dendritic cells. A combination of GM-CSF (10 ng/ml) and IL-4 (10-20 ng/ml) has also been found to be useful with this invention. It may also be desirable to vary the concentration of cytokines at different stages of the culture such that freshly cultured cells are cultured in the presence of higher concentrations of IL-4 (1000 U/ml) than established cultures (500 U/ml IL-4 after 2 days in culture). Other cytokines such as IL-13 may be substituted for IL-4.

The cultured immature dendritic cells typically do not label with mAb markers found on mature dendritic cells. Examples of markers for mature dendritic cells include, expression of surface CD83, DC-LAMP, p55, CCR-7, and expression of high levels of MHCII and costimulatory molecules, such as, for example, CD86 (Reviewed in, Banchereau and Steinman, 1998). Immature dendritic cells are identified based on typical morphology, expression of lower levels of MHC II and costimulatory molecules, and the lack of expression of DC maturation markers, e.g., surface expression of CD83 and expression of DC-LAMP, and lack of CD14 expression. In addition, examples of positive markers for immature dendritic cells include, but are not limited to, DC-SIGN (Geijtenbeek et al., 2000), intracellular CD83 (Albert et al., 1998), Langerin, and CD1A.

Thus, by utilizing standard antibody staining techniques known in the art, it is possible to assess the proportion of immature dendritic cells in any given culture. Antibodies may also be used to isolate or purify immature dendritic cells from mixed cell cultures by flow cytometry or other cell sorting techniques well known in the art.

### Contacting Immature Dendritic Cells with Antigen

Immature dendritic cells are contacted with an antigen or antigens for which reduction of an immune response is desired. The antigen may be any antigen against which antigen-specific T cells already exist. Among the preferred antigens are antigens relating to autoimmune diseases and organ transplant rejection. Examples of autoimmune diseases include, but are not limited to, juvenile diabetes, multiple sclerosis, myasthenia gravis, psoriasis, lupus, and atopic dermatitis. Examples of candidate antigens for some of these diseases include insulin and glutamic acid decarboxylase (GAD), and islet associated autoantigen in diabetes, myelin basic protein and proteolipid protein in multiple sclerosis, acetylcholine receptor in myasthenia gravis, and nuclear and ribosomal proteins, as well as nucleic acid protein complexes, such as histones, in lupus. Included among the autoantigens are those derived from stem cells, or whole cell preparations from cell lines such as insulinoma, thymic tissue, B lymphoblastoid cells, or cells such as pancreatic beta cells which are generated from stem cells.

Without being bound by theory, it is believed that autoimmune diseases result from an immune response being directed against “self-proteins”, i.e. autoantigens that are present or endogenous in an individual. In an autoimmune response, these “self-proteins” are being presented to T-cells which cause the T-cells to become “self-reactive”. According to the method of the invention, immature dendritic cells are contacted with the endogenous antigen, preferably during cell culture in vitro, and take up the antigen, so that when the immature dendritic cells are administered to a subject they have the capacity to specifically “turn-off”, i.e., “silence”, the pre-existing self-reactive T cells.

Other examples of pre-existing T cell responses to be silenced by the immature dendritic cells of the invention include T cells of different subsets, such as TH1 and TH2 CD4+ helper cells and CD8+ killer cells, as well as T cells at different stages of differentiation, such as naive T cells and especially, already formed CD4+ helper T cells and CD8+ killer T cells.

In addition, the immature dendritic cells administered according to the invention are able to stimulate the production of regulatory IL-10 producing T cells that also specifically silence or suppress the pre-existing T cells.

Similarly, the immature dendritic cells of the invention can be used to inhibit pre-existing T cells in the case of organ transplantation, where the organ recipient rejects the transplanted organ. According to the method of the invention, immature dendritic cells can be contacted with antigen derived from the organ or organ donor and administered to the organ recipient on or after transplantation to silence or suppress antigen specific T cells and facilitate organ graft acceptance. For example, dendritic cells may be co-cultured with live or dead cells from the organ or defined antigens derived from the organ. Or alternatively, cells may be coated with antibodies as a way of delivering them to dendritic cells.

In one embodiment of the invention, cultures of immature dendritic cells are contacted with the antigen of interest on or about day 3-6 of culture for a time sufficient to allow the antigen to be taken up by the immature dendritic cells. The duration of antigen exposure can vary, but is, typically, less than 1-2 days. The amount of antigen used, as well as the day on which the immature dendritic cells are contacted with antigen, can vary depending on the specific antigen of interest. Those skilled in the art may employ conventional clinical and laboratory means to optimize the effectiveness of the immature dendritic cell system. In a majority of cases, the immature dendritic cells, on day 4-7 of culture, are administered within a day or two after contact with generally 0.1-10 ug/ml of the antigen of interest. At this time (days 4-7), when DCs are still relatively immature, the cells may be modified (for example, using vectors expressing IL-10), to help maintain them in an immature state. Alternatively, the immature dendritic cells can be cryopreserved and thawed for use in tolerizing vaccines, or lyophilized and reconstituted for ease of use in therapeutic kits.

Compositions and Administration of Immature Dendritic Cells

When contacting immature dendritic cells with antigen in vitro, the immature dendritic cells are washed free of antigen and resuspended in a pharmaceutically acceptable carrier before administration to a mammal. Depending on the route of administration, different pharmaceutically acceptable carriers may be used. The dendritic cells of the invention may be administered in solution for intravenous, subcutaneous, intramuscular, or intraperitoneal administration. Preferably, the immature dendritic cells are administered subcutaneously.

For subcutaneous administration, the immature dendritic cells can be suspended in saline, plasma, serum or another suitable vehicle at physiological pH as are well known to those skilled in the art.

For intravenous administration, the immature dendritic cells can be suspended in a saline solution containing an appropriate concentration of plasma. Other examples of pharmaceutically acceptable carriers for intravenous use include, but are not limited to, cell culture medium or buffered saline.

Therapeutically effective concentrations of immature dendritic cells may range from about 1×40×10⁶ immature dendritic cells per single dose. The preferred dosage range is between 2 to 20×10⁶ immature dendritic cells per dose. Multiple administrations are also contemplated by the invention in order to sustain, or enhance, the therapeutic effect of the immature dendritic cells. Those skilled in the art will recognize that the dosage ranges and numbers of administrations will depend on such factors as the route of administration, the specific antigen of interest, and/or the effects of each injection.

In yet another embodiment of the invention, the immature dendritic cells of the invention can be included in a kit for use in inhibiting and/or suppressing antigen specific T cell function in vivo. The immature dendritic cells may be isolated in accordance with the methods described herein. The immature dendritic cells may be stored in frozen or lyophilized forms. Antigens may also be included in the kits when the immature dendritic cells have not yet been contacted with antigen. Antigens may be in any form, including, but not limited to, protein, DNA, RNA, and reconstituted in liposomes.

In another embodiment, rather than using whole immature dendritic cells, the kits may comprise immature dendritic cell membrane fragments with or without preloading with antigen, or alternatively, for example, liposomes containing reconstituted immature dendritic cell molecular components sufficient to silence or suppress an antigen-specific T cell response in vivo. The kit may also include other immunosuppressive agents and pharmaceutically acceptable carriers, or any other number of elements which would make the kit convenient and easy to use, and facilitate the use of the immature dendritic cell system in a clinical setting.

In a further embodiment, immature dendritic cells can be contacted in vivo with an antigen of interest. Antigens can be targeted to the sites of immature dendritic cells in vivo, because the immature dendritic cells are preferentially involved in antigen uptake by receptor mediated pathways, e.g., through Fc receptors, through lectins like Langerin and DC-SIGN, through multilectins like DEC-205 and the macrophage mannose receptor, and through receptors for dying cells.

In this embodiment, progenitors of dendritic cells, which are resident in tissue, can be pretreated with cytokines such as FLT-3 or G-CSF in order to increase the number of immature dendritic cells. Additionally, cytokines such as GM-CSF and IL-4 may be administered to further enrich the population of immature dendritic cells. Thus, in this embodiment, dendritic cells are enriched in vivo, and the immature dendritic cells are then contacted with antigen in vivo by administering the antigen. Preferred routes of administration of antigen include intravenous, intramuscular, and subcutaneous. More preferred are intravenous and subcutaneous.

Once immature dendritic cells are contacted with the antigen in vivo, they are capable of silencing and/or suppressing existing CD8+T cell function. This process of antigen specific silencing may involve induction of regulatory T cells directly in vivo or transfer of antigen to a specialized dendritic cell with immunosuppressive properties resident in the lymphoid tissue.

A further embodiment relates to modification of immature dendritic cells prior to injection to keep them in an immature state and prevent spontaneous maturation in vivo, for example, by transforming the immature dendritic cells with at least one vector comprising a gene encoding TGF-β and/or IL-10 family proteins, preferably IL-10, or with RNA encoding these cytokines. (Moore et al. 1993). Examples of such vectors include, but are not limited to, vaccinia virus or adenovirus, that have been shown to infect dendritic cells (see, U.S. Pat. No. 6,300,090). This dendritic cell modification may be performed concurrently with antigen loading.

Another embodiment relates to the use of immature dendritic cells to generate antigen specific regulatory CD8+T cells in vitro, which may then be used for adoptive immunotherapy in vivo. In this system, T cells are co-cultured with immature dendritic cells in vitro at a dendritic cell to T cell ration of about 1:10-100, and the resulting T cells are then injected for the purpose of suppressing an active immune response. The preferred route of administration of such T cells is intravenous. The dose of T cells injected may vary (1-100×10⁶ cells), but the usual dose may be about 10-20×10⁶ cells. Multiple injections are also contemplated in this embodiment.

The following non-limiting examples serve to illustrate certain specific embodiments of the invention in more detail.

### EXAMPLES

### Example 1

### Antigen Specific Inhibition of Effector T Cell Function in Humans after Injection of Immature Dendritic Cells

To examine whether ex vivo maturation stimulus is essential for the immune efficacy of a dendritic cells (DC) vaccine, we initiated a clinical study comparing DCs cultured with or without such a stimulus. DCs were pulsed with Keyhole Limpet Hemocyanin (KLH) and in the case of HLA A2.1+ subjects, additionally with HLA A*0201 restricted influenza matrix peptide (MP). Here we describe the findings on the first two study subjects injected with immature DCs.

### METHODS

Study Desiqn

The study was initiated as a randomized 2×2 factorial design, comparing a single injection of immature versus mature DCs administered subcutaneously (s.c.) versus intradermally (i.d.). The inhibition of antigen specific effector function in the first 2 subjects (Im1 and Im2) injected s.c. with immature DCs is described. Two additional subjects received an injection of mature DCs, either i.d. (M1), or s.c. (M2).

Human Volunteers

Normal healthy adult volunteers were recruited through advertisement. Eligibility and exclusion criteria were as in a prior study, Dhodapkar et al. 1999, and included age 18-65 years and no clinical evidence of malignancy, chronic infection or autoimmunity. All subjects signed an informed consent and the study was approved by the Rockefeller University Institutional Review Board.

Baseline studies

All study participants were typed for HLA A2.1 status, and initially followed for a 1-3 month period during which at least 2 baseline measurements of immune response were made. Laboratory tests at baseline to confirm eligibility included complete blood count, chemistry profile, hepatitis B, C and HIV serology, rheumatoid factor, antinuclear antibody (ANA), urinalysis, chest X ray, pregnancy test, influenza serology and anergy panel consisting of candida, mumps and tetanus.

Generation and injection of dendritic cells

DCs were generated from plastic adherent blood monocyte precursors following in vitro culture with GM-CSF and IL-4 as described, Dhodapkar et al., 1999, and pulsed with antigens on day 5 of culture. The antigens were: 10 μg/ml KLH (depyrogenated, Calbiochem), and 1 μg/ml influenza MP (manufactured in the microchemistry facility of the Sloan Kettering Institute by Dr A Houghton). Autologous monocyte conditioned medium (50% v/v) was added on day 5 of culture as a maturation stimulus for subjects receiving mature DCs (M1, M2), Dhodapkar et al., 1999, U.S. application Ser. No. 08/600,483 and WO 97/29182, which are herein incorporated by reference. Two million DCs were injected on day 6 (Im1) or day 7 (Im2, M1, M2) of culture (Table 1). On the day of injection, the DCs were washed free of antigen, resuspended in normal saline containing 5% autologous plasma in two 0.1-0.2 cc aliquots and injected within 30 minutes of final reconstitution. The phenotype of the injected DCs was monitored by flow cytometry. All injected DC preparations tested negative for bacterial and fungal contamination.

Follow up and monitoring

Immune responses were evaluated 1 week after DC injection and at 1-3 month intervals thereafter. Both subjects had a repeat hemogram, rheumatoid factor, antinuclear antibody and influenza serology 1 month after DC injection.

Measurement of Immune responses

Measurement of immune response was performed on freshly isolated blood mononuclear cells (PBMCs). In addition, for most assays, cryopreserved pre and post-immunization specimens were thawed, coded and assayed together in a blinded fashion.

ELISPOT assay for antigen specific cytokine (IFNγ, IL-4, IL-10) secreting T cells

Antigen specific T cells were quantified using a standard ELISPOT assay as described, Dhodapkar et al., 1999, after overnight culture in the presence or absence of antigens in plates precoated with anti-cytokine (IFN-γ, IL-4 or IL-10) antibodies (Mabtech, Stockholm). Antigens were 1 μg/ml HLA A*0201 restricted peptides from influenza matrix protein (MP, GILGFVFTL), HIV gag (gag, SLYNTVATL) and cytomegalovirus pp65 (CMV, NLVPMVATV) as controls. The background reactivity with no peptide in this assay was low (mean±SE: 1+1 spot forming cells/2×10⁵ cells). For the detection of influenza specific responses, PBMC were infected with influenza virus at a multiplicity of infection (MOI) of 2. For some assays, MP pulsed mature DCs were used as APCs (PBMC: DC ratio 30:1). KLH (10 μg/ml) was also used as an antigen (no protein and superantigen as controls) in some assays. In additional assays, bulk T cells were depleted of CD4+ and CD8+T cells using magnetic beads (Miltenyi, Bergisch-Gladbach, Germany) before use in the ELISPOT assays.

MHC tetramer binding assays

Soluble influenza MP-HLA A*0201 tetramers were prepared as described, Busch et al., 1998 and binding to tetramers was analyzed by FACS analysis. Frozen aliquots of PBMC from pre and post immunization were thawed together and stained as described Dhodapkar et al., 1999, with A*0201-MP tetramer at 37° C., both directly and after 7 day co-culture with autologous MP pulsed DCs (unpulsed DCs as controls).

Recall T cell assays

For recall assays, pre/post immunization PBMCs were co-cultured with freshly generated autologous mature DCs pulsed with MP (unpulsed DCs as control) at PBMC:DC ratio of 30:1 for 7 days. At the end of 7 day culture, MP specific T cells were quantified as described earlier, Dhodapkar et al., 2000, using: a) ELISPOT assay for antigen specific cytokine producing cells (after restimulation on day 7); b) MHC-tetramer binding assay; or c) CTL assay. CTL activity was measured in a standard 5 hour⁵¹Cr release assay at (effector:target) E:T ratio of 20:1. Targets were T2 lymphoblastoid cells pulsed with 1 μg/ml MP, or unpulsed T2 cells as controls. Excess cold K562 cell targets (80:1) were used to inhibit NK mediated lysis.

Antigen dependent proliferation:

Antigen dependent proliferation assays were performed as described, at 2 PBMC dose levels (3×10⁴ cells/well and 1×10⁵ cells/well) in the absence or presence of graded doses of KLH (0.1-10 μg/ml), Dhodapkar et al., 1999. Tetanus toxoid (TT) and staphylococcal enterotoxin A (SEA) served as control antigens. For some assays, bulk T cells were depleted of CD4+ and CD8+T cells using magnetic beads (Miltenyi) before use in proliferation assays.

### RESULTS

Inhibition of MP specific effector T cell function in vivo after DC injection:

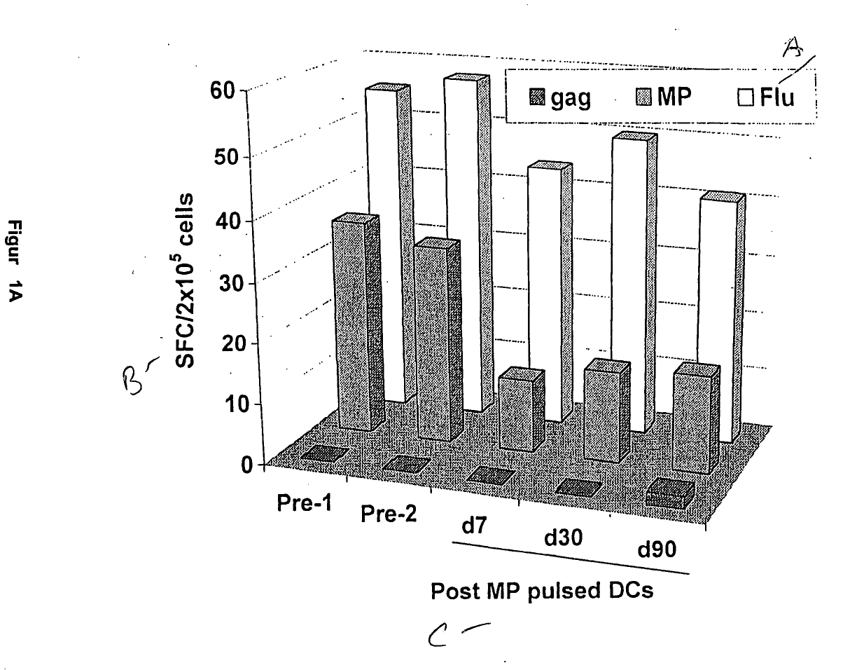

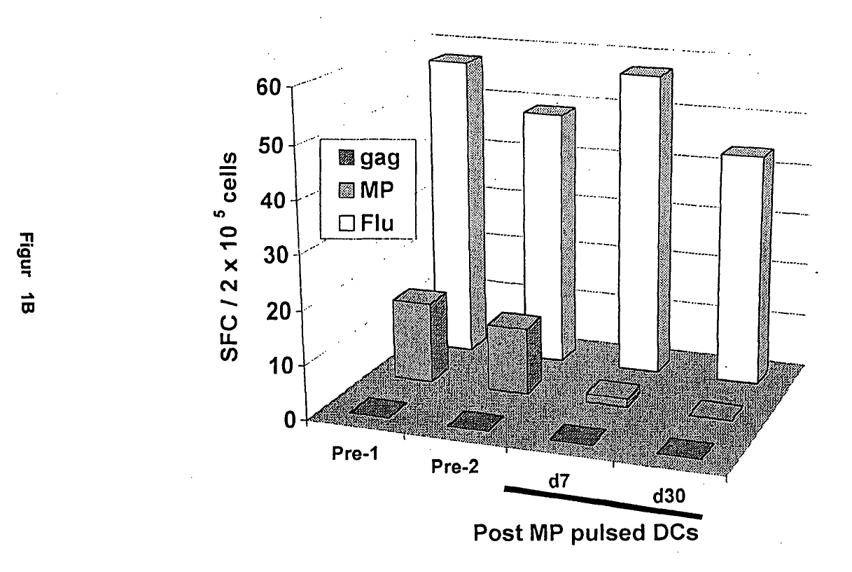

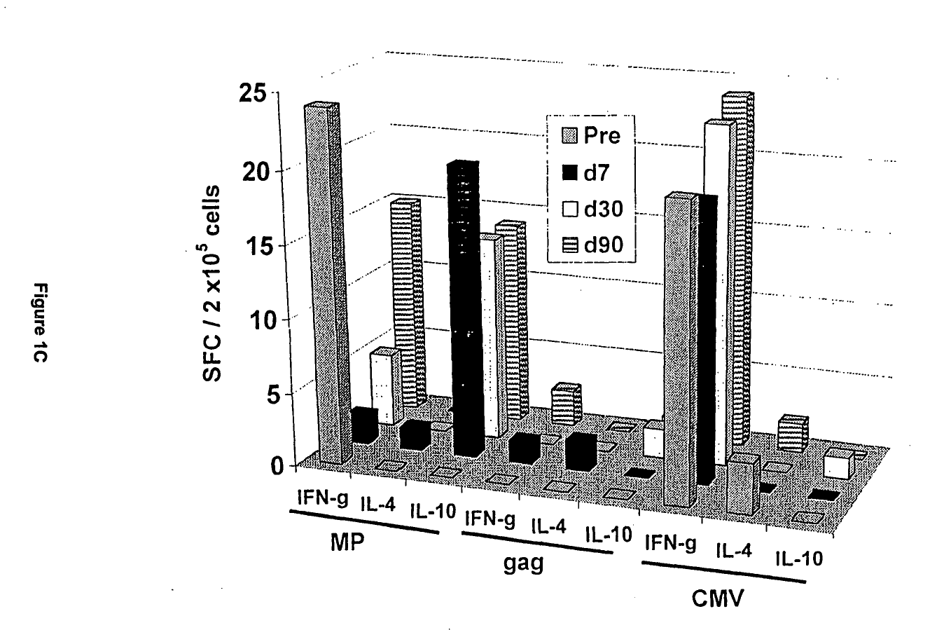

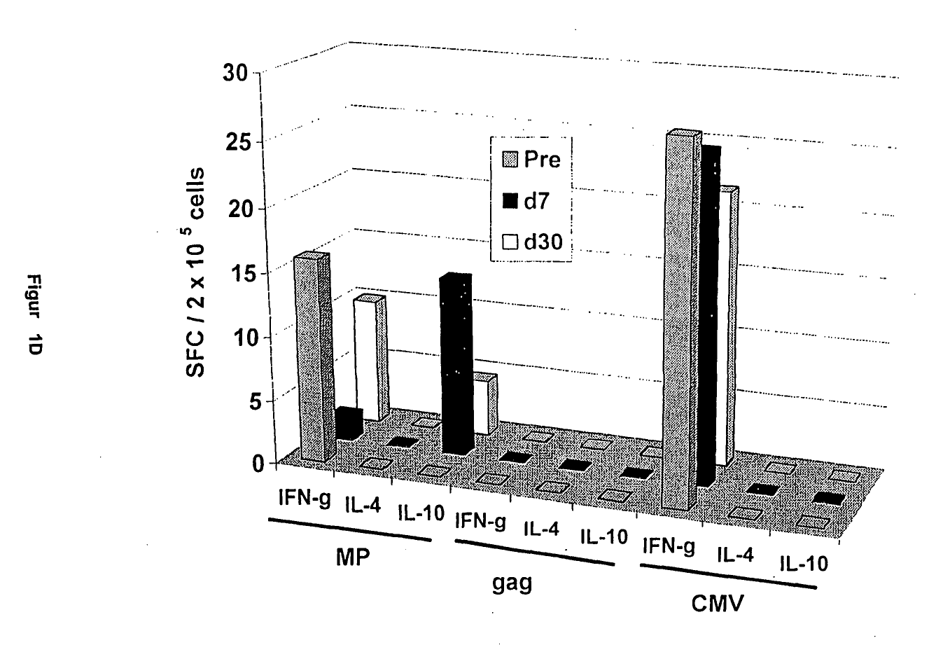

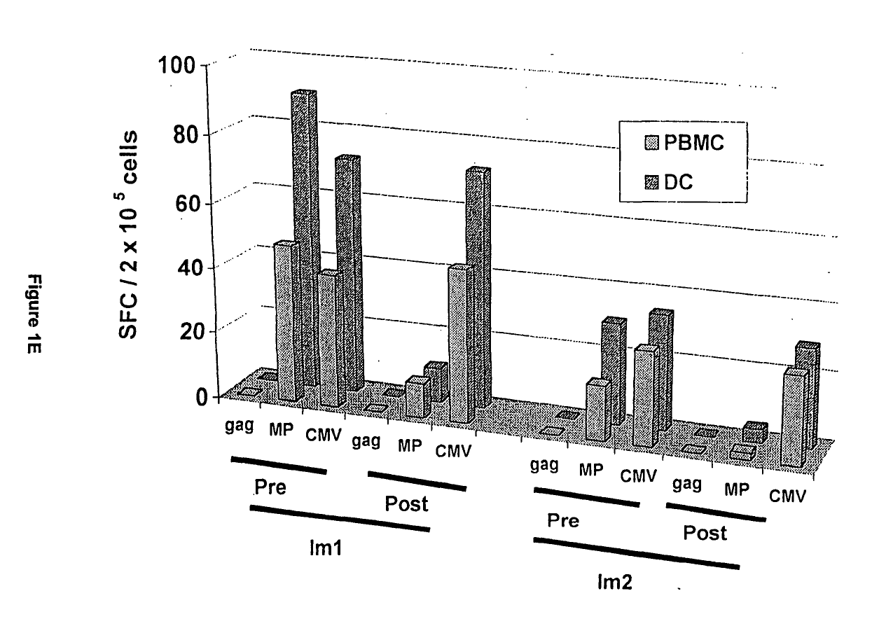

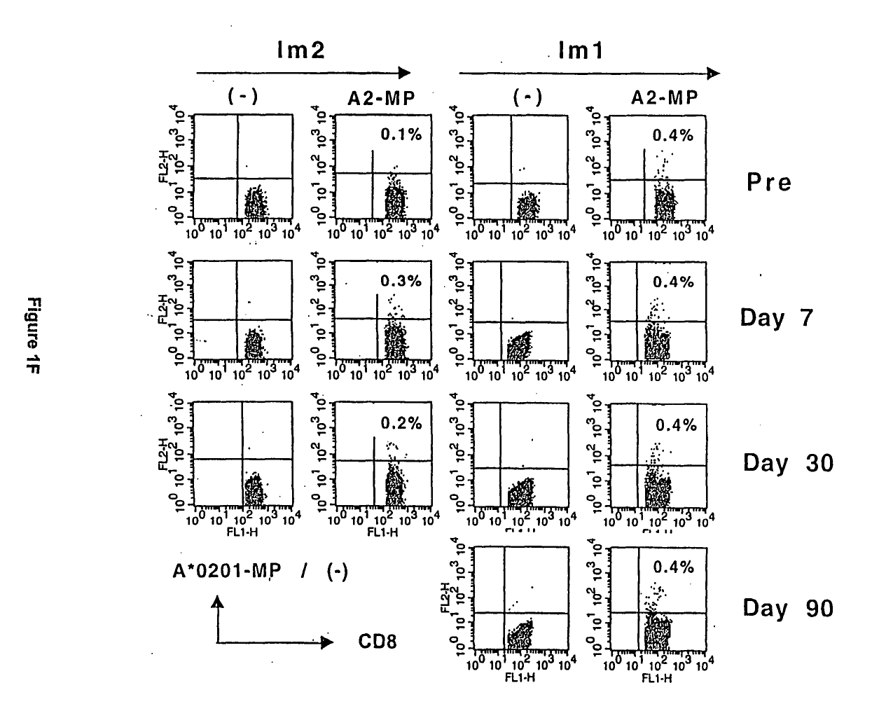

Two subjects (Im1 and Im2) received a single s.c. injection of 2×10⁶ immature DCs pulsed with the influenza peptide, MP, and KLH (Table 1). All DC injections in this study were well tolerated without any clinical toxicity and serologic/clinical evidence of auto-immunity. Before immunization, MP specific interferon-γ (IFN-γ) producing T cells were detectable in both subjects as expected, because most adults have been exposed to the influenza virus. However, after DC immunization, there was a decline in MP specific IFN-γ producing cells (FIG. 1A and 1B). The number of MP-specific effectors reached a nadir 7-30 days post immunization and improved thereafter. In contrast, there was little decline in total influenza-effector T cell function, indicating that the decrease in MP-effector function was specific for the immunizing peptide. Similar data were obtained when cryopreserved cells were assayed together (FIG. 1C and D), although the absolute reactivity was higher with fresh cells, as reported previously, Dhodapkar et al., 1999. As a control, no decline in antigen specific IFN-γ producing cells to HLA A*0201 restricted CMV peptide was observed. The loss of MP specific IFN-γ producing cells persisted, even when peptide pulsed mature DCs were utilized as APCs in the ELISPOT (FIG. 1E). In fact, the use of DCs increased the pre-immunization measurements, so that the decrease in effectors after immature DC injection was even more striking.

Induction of antigen specific IL-10 producing cells in vivo:

The decline in MP specific IFN-γ producing cells was associated with the appearance of MP specific IL-10, but not IL-4 producing T cells (FIG. 1C and D). No induction of IL-4/IL-10 producing cells to the control antigen CMV, was observed. The preimmunization IFN-γ secretors and post immunization IL-10 producers, were both CD8+ CD4-, as indicated by magnetic bead depletion experiments (not shown). MP specific CD8+T cells elicited after mature DC injection in an earlier study, Dhodapkar et al., 1999, failed to produce IL-10 (not shown), so we propose that the induction of IL-10 producers is due to the use of immature DCs.

Decline of effectors is not due to loss of circulating MP specific T cells:

The decline in MP (but not CMV or influenza specific effectors) after immunization with MP pulsed immature DCs raised several possible mechanisms: immature DCs could lead to the loss of circulating MP specific T cells due to either cell death/deletion, or these cells may redistribute to tissues/nodes after activation in vivo. Alternatively, DC injection could lead to inhibition of effector T cell function or induction of anergy in an antigen specific manner. Analysis of MP specific T cells by MHC tetramer binding in uncultured PBMC revealed either no change (Im1) or an increase (Im2) in MP specific T cells post DC immunization (FIG. 1F). Therefore the loss of effector function after immunization with immature DCs is not due to a loss of circulating antigen specific T cells.

Expansion of memory T cells with defective effector function in vivo:

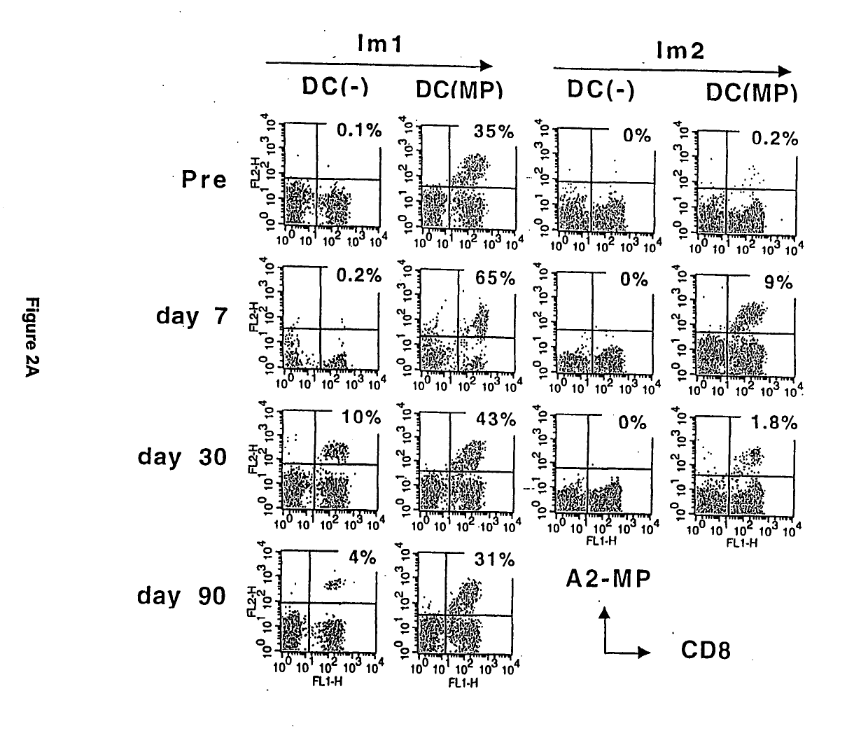

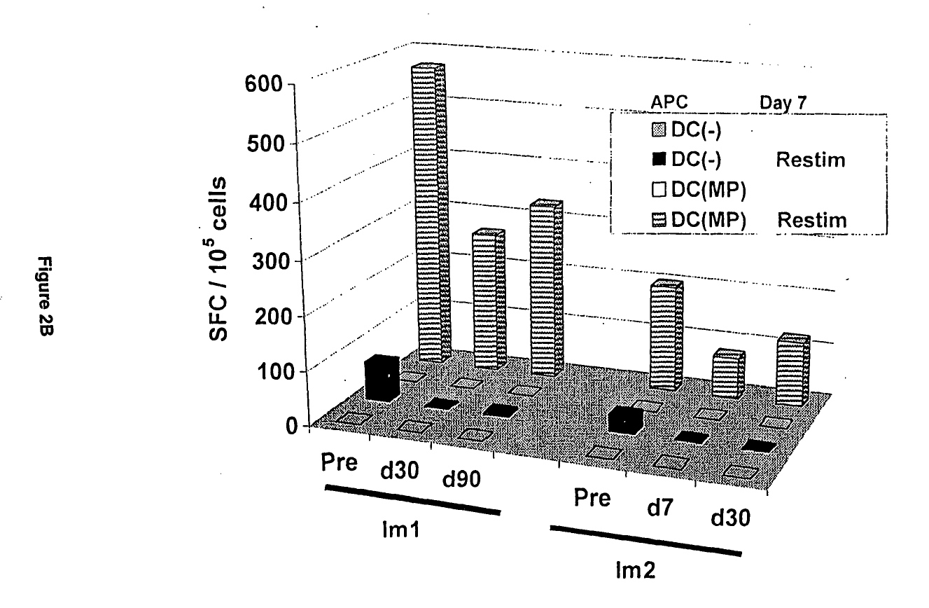

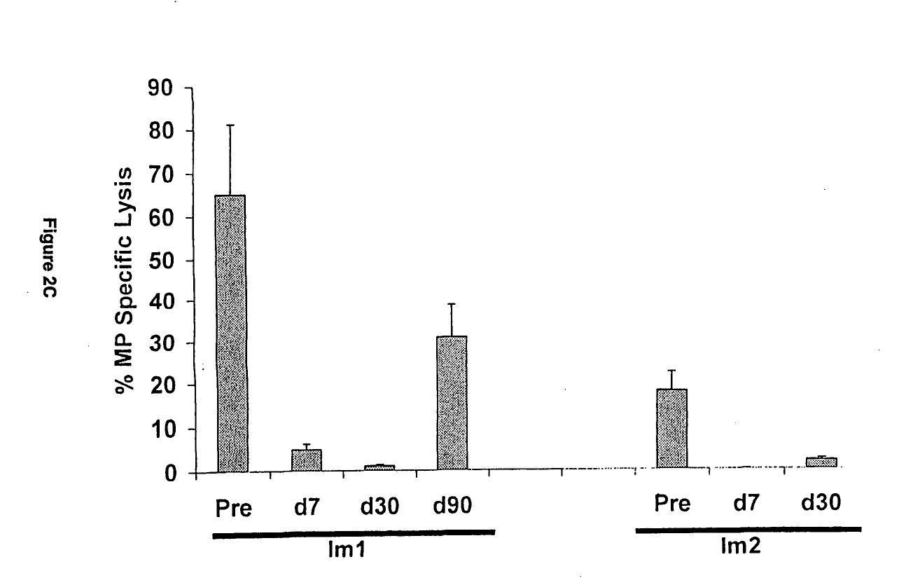

When pre and post immunization samples were thawed together and boosted in culture with MP pulsed autologous mature DCs, there were greater number of MP specific MHC tetramer binding T cells in post-immunization cultures (FIG. 2A). However, these specific antigen-binding T cells had reduced MP specific IFN-γ producers in the ELISPOT assay (FIG. 2B) and failed to kill peptide pulsed targets (FIG. 2C), even when they constituted up to 60% of all CD8+T cells. There was no expansion of MP specific IL-10 or IL-4 producing T cells in these cultures (not shown). We conclude that immunization with immature antigen bearing DCs blunts the capacity of the corresponding antigen-specific CD8+T cell to mount lytic function in vitro.

Priming of KLH specific T cells in vivo:

TABLE 1
Characteristics of dendritic cells

| | HLA | | Dendritic Cell | | | % Large cells expressing | | |
|---|---|---|---|---|---|---|---|---|
| ID | A2.1 | Antigens | Maturity | Route | Purity* (%) | HLADR+ | CD83+ | CD14+ |
| Im1 | (+) | KLH, MP | Immature | s.c. | 87 | 91 | 7 | 2 |
| Im2 | (+) | KLH, MP | Immature | s.c. | 76 | 100 | 3 | 1 |
| M1 | (−) | KLH | Mature | i.d. | 87 | 99 | 93 | 1 |
| M2 | (−) | KLH | Mature | s.c. | 96 | 100 | 92 | 0 |

*percent dendritic cells by morphology. Dose of DCs injected in all subjects (after accounting for purity) was 2 × 10⁶ DCs.
NE: not examined.

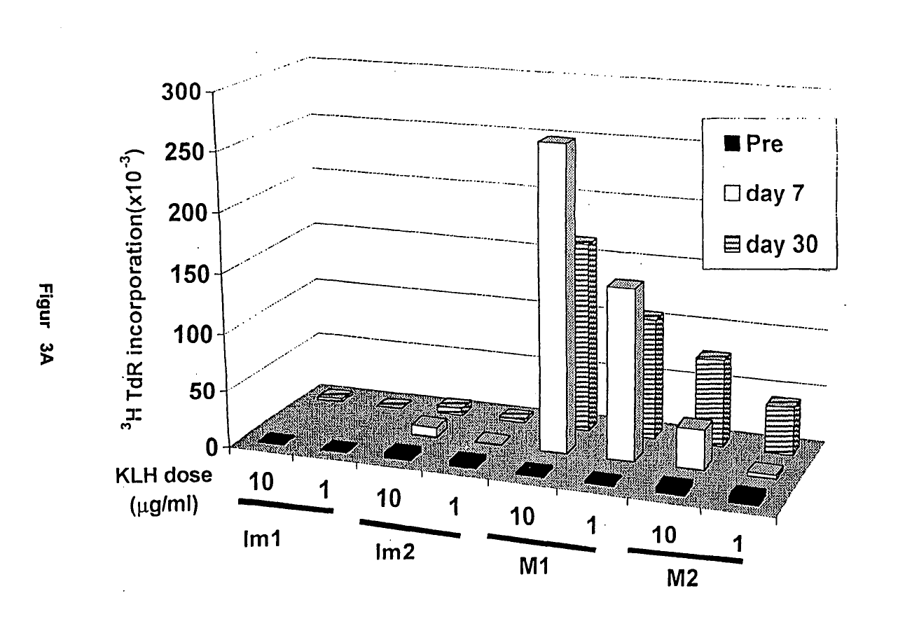

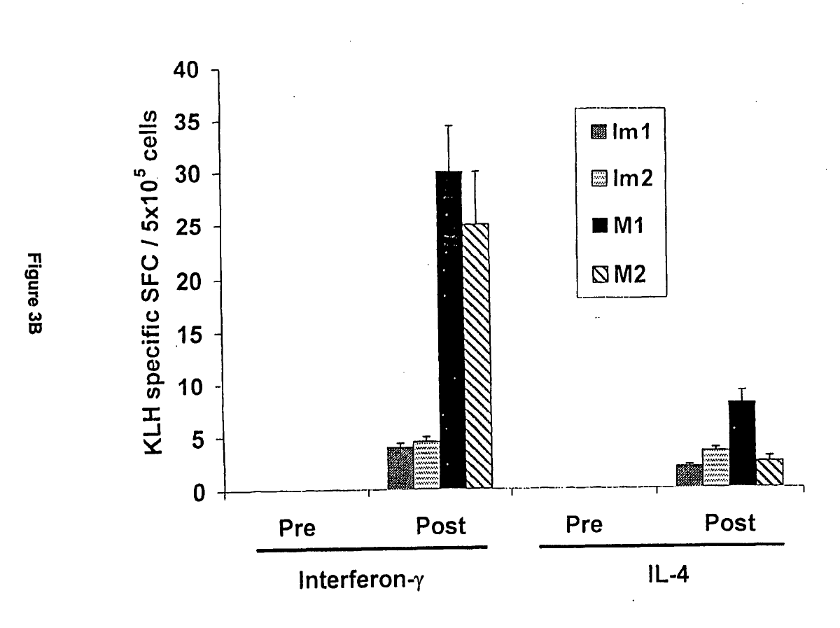

The two volunteers immunized with mature DCs (M1, M2) were not HLA-A2.1 positive and therefore could not be studied for their response to the influenza MP peptide. However, cryopreserved samples of PBMCs from all volunteers were assayed together for KLH-specific proliferative responses. All samples showed strong responses to a superantigen used as a positive control, but the KLH priming was much greater when mature DCs had been used (FIG. 3A). Small proliferative responses to KLH were noted in freshly thawed specimens following immature DC injections (not shown), but no KLH specific interferon-γ secreting cells were evident, in contrast to clear Th1 priming with mature DCs (FIG. 3B). Therefore the primary CD4 T cell responses were weaker when immature DCs were used to immunize to KLH.

Example 2

Antigen-bearing, immature dendritic cells induce peptide-specific, CD8+ regulatory T cells in vivo in humans

Regulatory T cells (T_{R}) can suppress the function of other effector T cells in the setting of autoimmunity, transplantation, and resistance to tumors. T_{R} have been clearly identified in mice and humans (Roncarolo et al, 2000; Waldmann et al, 2001; Sakaguchi S, 2000). These T_{R} can inhibit strong responses mediated by CD4+ and CD8+ effector T cells, preventing allograft rejection, graft versus host disease, chronic inflammatory disease and autoimmunity (Reviewed in, Roncarolo et al, 2000; Waldmann et al., 2001; Sakaguchi S, 2000). Recent studies have identified T_{R} in human blood, where they have two main functional properties (Taylor PA et al., 2001; Dieckmann et al, 2001; Levings et al., 2001). First, they proliferate poorly in response to mitogenic stimuli. Second, they can dampen the responses of effector T cells (Shevach, E. M., 2001). Although most studies have characterized CD4+ T_{R} (Groux et al., 1997; Sakaguchi et al., 1995), CD8+T cells with regulatory properties have also been described (Gaur et al., 1993; Koh et al., 1992; Borthwick et al, 2000; Koide et al. 1990; Colovai et al., 2001; Filaci et al., 2001; Balashov et al., 1995).

Certain populations of T_{R}, particularly those expressing CD4 and the CD25 IL-2 receptor chain, are generated in the thymus, where the cortical epithelium was recently identified as a critical antigen presenting cell (Bensinger et al., 2001). T_{R}, often identified by their capacity to produce IL-10, can also be induced peripherally in the settings of transplantation and graft versus host disease (Roncarolo M G, Levings M K, 2000; Waldmann H, Cobbold S, 2001), but the antigen presenting cell (APC) requirements have not been identified. It is important to identify pathways that control the formation of T_{R}, since these would provide novel strategies for antigen specific immune-suppression or immune tolerance.

Dendritic cells (DCs) are powerful APCs for the induction of effector T cells (Banchereau et al. 1998). In order to initiate immunity, DCs must carry out two sets of linked events (Mellman et al., 2000). One is the capture of antigens and successful formation of MHC-peptide complexes; the second is to undergo a process termed “maturation”, acquiring many additional properties to stimulate immunity (Mellman et al., 2000). Immature DCs appear to be inactive as inducers of immunity in vivo (Inaba et al., 2000). However in a standard tissue culture assay involving initiation of the mixed leukocyte reaction, immature DCs were not inactive but instead induced the formation of T_{R}, with both the anergic and regulatory properties mentioned above (Jonuleit et al., 2000). In the previous example, we tested the effects of immunizing volunteers with immature DCs. Example 1 illustrates the findings on two healthy volunteers who received a single subcutaneous injection of 2×10⁶ immature DCs pulsed with an HLA A*0201 restricted influenza matrix peptide (MP). In contrast to prior findings using mature DCs (Dhodapkar et al., 1999), injection of immature DCs was associated with antigen specific inhibition of effector T cell function. The peptide specific IFNγ-producing cells disappeared from the blood, and cytolytic cells could no longer be expanded in culture. However, antigen binding CD8+T cells were still present, and circulating MP specific IL-10 producers developed. In summary, the use of immature DCs silenced effector T cell functions, raising the possibility that T_{R} were being induced in vivo.

The data which follows illustrates the capacity of immature DCs to induce antigen specific regulatory CD8+T cells in humans.

### METHODS

Study design and DC injection:

This study describes the findings on 2 subjects (Im1 and Im2), injected s.c. with immature DCs derived by culture of blood monocyte precursors in GM-CSF and IL-4, as described in Example 1. The injected DCs were pulsed with keyhole limpet hemocyanin (KLH) and influenza MP during the last 16 hrs of a 6 day (Im1) or 7 day (Im2) monocyte culture as described in Example 1.

Follow up and immune monitoring:

All subjects were evaluated 1 week after DC injection, and at 1-3 month intervals thereafter. Both subjects had normal repeat hemograms, and absent rheumatoid factor and antinuclear antibody, 1 and 3 months after DC injection. Antigen specific T cells were quantified using a standard ELISPOT assay for the presence of peptide specific IFN-γ, IL-4 or IL-10 producing cells (Dhodapkar et al, 2001). For cytolytic T lymphocyte (CTL) assays, T cells were cocultured with peptide pulsed mature DCs for a week, before measurement of lytic activity, as described (Dhodapkar et al., 2001). DC maturation was achieved by 1 day of culture in a mixture of IL-1β, IL-6, TNFα and PGE₂.

Assays for regulatory cells:

PBMCs from 7 days post immunization (T_{R} sample) and either pre immunization or recovery (e.g. day 180) time points were thawed and cultured (2-3×10⁵ cells/well) either separately or together, in the presence of peptide pulsed autologous monocyte derived mature DCs at PBMC:DC ratio of 60:1, in ELISPOT plates precoated with anti-interferon-γ antibody (Mabtech). After overnight culture, the number of antigen specific interferon-γ producing cells was determined by ELISPOT assay, as described previously (Dhodapkar et al., 1999). In addition to the immunizing peptide (Flu matrix peptide or MP, GILGFVFTL), additional control HLA A*0201 restricted peptides were from EBV LMP-2 (CLGGLLTMV) and HIV-1 gag (SLYNTVATL).

In one subject (Im2), sufficient numbers of cells from the day 7 post immunization sample were available to further characterize T cell mediated suppression. In this subject, dose dependence was tested with graded doses (1:1, 1:10) of day 7 PBMC to pre-immune or convalescent (d180) PBMC. Also, T_{R} containing PBMCs (from day 7) were depleted of CD8+T cells by immunomagnetic beads (Miltenyi), before adding to the co-cultures. For some experiments, the T_{R} samples were separated from the recovery specimens by a transwell to check for soluble suppressor factors. In these cultures, APCs were added on either side of the transwell. In some experiments, the co-cultures of T_{R} and recovery cells were performed in the presence of neutralizing anti-IL-10 antibody (10 μg/ml, 12G8, Genzyme, Cambridge, Mass.), or 100 U/ml of rIL-2 (Chiron).

### RESULTS

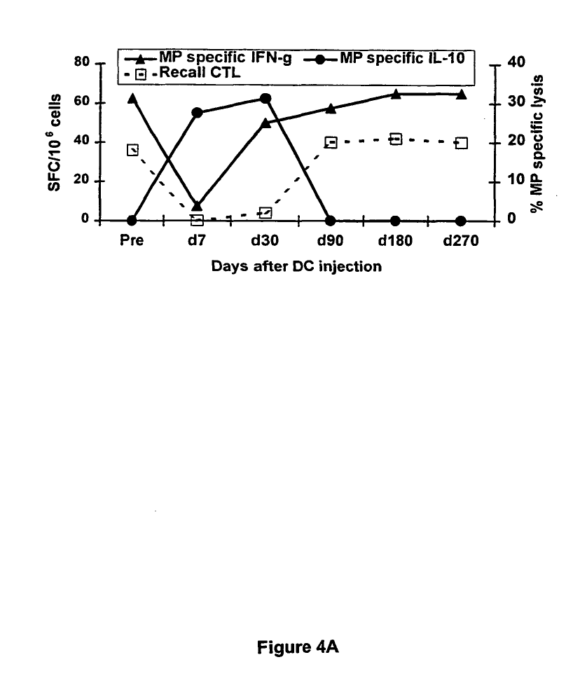

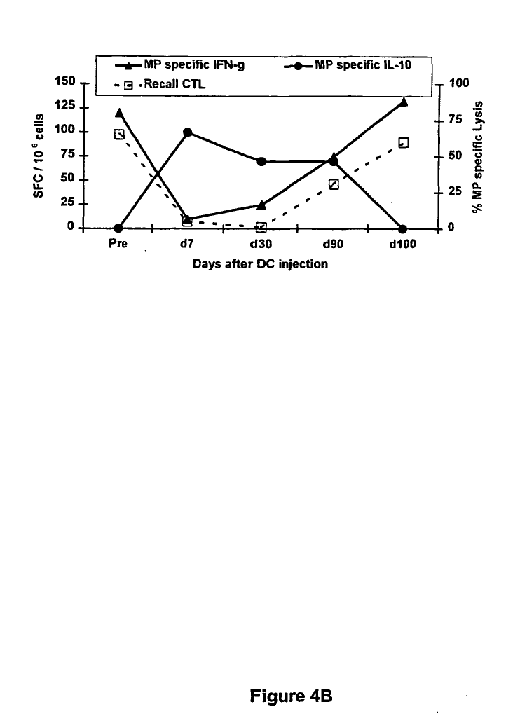

Both healthy volunteers had been primed to influenza, because prior to immunization they had influenza MP-specific effector T cells according to two criteria: interferon-γ producing T cells were found in ELISPOT assays, and peptide specific CTLs could be expanded by a week of culture with mature DCs. However, 1 week after the injection of MP pulsed immature DCs, these effector functions were silenced in the blood. This loss of function was reversible, returning to pre-injection levels by 3-4 months post injection in both subjects (FIG. 4A and 4B). In a reciprocal fashion, silencing and recovery of effector T cell function were associated with the appearance and then decline in peptide specific IL-10 producers, which were no longer detectable after 90-100 days post immunization (FIG. 4). The DC injections were not associated with any clinical toxicity or clinical and serologic evidence of auto-immunity in both subjects. Thus, the inhibition of effector T cell function after a single injection of immature DCs is self limited.

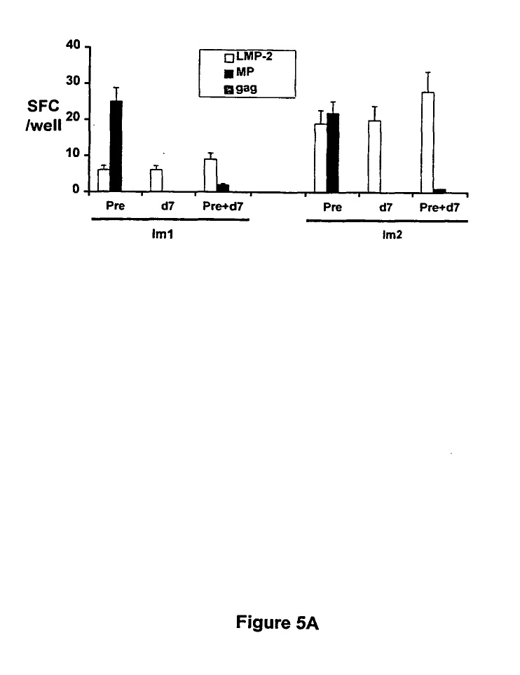

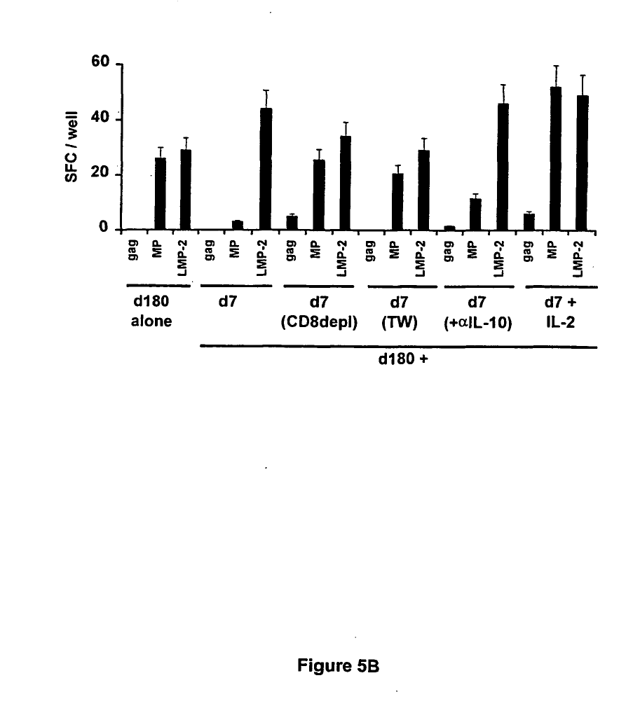

Because we had shown that the loss of circulating MP specific effector T cell function was not associated with a decline in circulating MHC tetramer binding cells (Dhodapkar et al., 2001), we tested if the effector silencing following injection of immature MP pulsed DCs was mediated by the induction of regulatory T cells. To directly test this, we mixed T cells from 1 week postimmunization (when the effector silencing was maximal), with either preimmunization or recovery samples. The day 7 PBMCs inhibited MP specific interferon-γ producers from pre-immunization cultures in both subjects (FIG. 5A). The inhibition was specific for the immunizing peptide, because the responses to the control peptide LMP-2 were not silenced. Thus peptide specific T_{R} are rapidly induced after the injection of immature peptide pulsed DCs in humans.

Further characterization of the suppression was only carried out in Im2, in whom we had additional cells available. The suppression of T cell function in these mixing experiments was dose dependent, and seen even at Tₛᵤₚₚᵣₑₛₛₒᵣ:T_{effector} ratio of 1:10 (not shown). Suppression was lost if day 7 cells were depleted of CD8+T cells, and also if cell contact between day 7 and recovery T cells was prevented in transwell cultures (FIG. 5B). Although the day 7 specimens had been shown to contain MP specific IL-10 producers, inhibition of IL-10 in these mixing experiments with neutralizing anti-IL-10 antibody led to only slight recovery of MP specific effectors (FIG. 5B). However, the suppression was fully reversed by the addition of 100 U/ml of IL-2 to these cultures (FIG. 5B). Thus peptide specific CD8+T_{R} cells induced in vivo by immature DCs inhibit CD8+T cells in a cell contact dependent manner, that is only partly IL-10 dependent.

These data provide direct evidence for the concept of antigen specific CD8+T cell mediated immune regulation, and the induction of such T cells in vivo in humans by immature DCs. Once induced, these cells have a limited life span in circulation. Thus, naturally occurring T_{R} may require continued antigen presentation by trafficking immature DCs. Because peptide specific IL-10 producing cells are also induced by immature DCs, we refer to these suppressor cells as T_{R}, in keeping with prior nomenclature. The regulation we observed required cell-cell contact and was only partly IL-10 dependent. These features are similar to the CD4+T_{R} cells induced by immature DCs in vitro (Jonuleit et al., 2000). A subset of CD8+ CD28- suppressor T cells has also been described, which mediates suppression in a cell contact dependent fashion (Borthwick et al., 2000; Koide et al., 1990; Colovai et al., 2001). The site where immature DCs generate T_{R} in vivo is not yet clear. Without being bound by theory, one possibility is that the DCs might traffic to lymph nodes to meet T cells recirculating via high endothelial venules. Another alternative, which is possible because T_{R} have an activated phenotype, is that the DCs activate T_{R} that circulate from blood to extravascular spaces (here the skin), and then return to the lymph node via the lymphatics.

Our data suggest DC maturation as a key therapeutic target for the regulation of immunity (Mellman and Steinman, 2001). The inhibition of maturation in antigen-capturing DCs may promote the induction of T_{R} cells in vivo. Impairment of CD8+T cell suppressor function has been observed in patients with human autoimmune diseases such as lupus and multiple sclerosis (Filaci et al., 2001; Balashov et al., 1995). A role for regulatory T cells has also been suggested for human allograft acceptance (VanBuskirk et al., 2000). In a reciprocal fashion, reduction of regulatory T cells may improve resistance to cancer and chronic infections, as noted in a recent study with experimental tumors in mice (Stutmuller et al., 2001).

All references cited herein are hereby incorporated by reference.

### REFERENCES

1. Appay, V., D. F. Nixon, S. M. Donahoe, G. M. Gillespie, T. Dong, A. King, G. S. Ogg, H. M. Spiegel, C. Conlon, C. A. Spina, D. V. Havlir, D. D. Richman, A. Waters, P. Easterbrook, A. J. McMichael, and S. L. Rowland-Jones. 2000. HIV-specific CD8(+) T cells produce antiviral cytokines but are impaired in cytolytic function. J Exp Med 192:63-76.

2. Austyn, J. M. 1987. Lymphoid dendritic cells. Immunol. 62, 161.

3. Balashov K E, Khoury S J, Hafler D A, Weiner H L: Inhibition of T cell responses by activated human CD8+T cells is mediated by interferon-gamma and is defective in chronic progressive multiple sclerosis. J Clin Invest. 1995; 95:2711-2719.

4. Banchereau J, Steinman R M: Dendritic cells and the control of immunity. Nature. 1998; 392:245-252

5. Banchereau, J., and R. M. Steinman. 1998. Dendritic cells and the control of immunity. Nature 392:245-252.

6. Bell, D., P. Chomarat, D. Broyles, G. Netto, G. M. Harb, S. Lebecque, J. Valladeau, J. Davoust, K. A. Palucka, and J. Banchereau. 1999. In breast carcinoma tissue, immature dendritic cells reside within the tumor, whereas mature dendritic cells are located in peritumoral areas. J Exp Med 190:1417-1426.

7. Bensinger S J, Bandeira A, Jordan M S, Caton A J, Laufer T M: Major histocompatibility complex class II-positive cortical epithelium mediates the selection of CD4(+)25(+) immunoregulatory T cells. J Exp Med. 2001; 194:427-438.

8. Borthwick N J, Lowdell M, Salmon M, Akbar A N: Loss of CD28 expression on CD8(+) T cells is induced by IL-2 receptor gamma chain signalling cytokines and type I IFN, and increases susceptibility to activation-induced apoptosis. Int Immunol. 2000; 12:1005-1013.

9. Buelens C, Verhasselt V, De Groote D, Thielemans K, Goldman M, Willems F. 1997. Human dendritic cell responses to lipopolysaccharide and CD40 ligation are differentially regulated by interleukin-10, Eu J Immunology, 27:1848-52.

10. Busch, D. H., I. M. Pilip, S. Vijh, and E. G. Pamer. 1998. Coordinate regulation of complex T cell populations responding to bacterial infection. Immunity 8:353-362.

11. Caux, C., C. Massacrier, B. Vanbervliet, B. Dubois, C. Van Kooten, I. Durand, and J. Banchereau. 1994. Activation of human dendritic cells through CD40 cross-linking. J Exp. Med. 180:1263-1272.

12. Caux, C., C. Massacrier, C. Dezutter-Dambuyant, B. Vanbervliet, C. Jacquet, D. Schmitt, and J. Banchereau. 1995b. Human dendritic Langerhans cells generated in vitro from CD34+ progenitors can prime naïve CD4+T cells and process soluble antigen. Immunol 155, 5427.

13. Cella, M., F. Sallusto, and A. Lanzavecchia. 1997. Origin, maturation and antigen presenting function of dendritic cells. Curr Opin Immunol 9:10-16.

14.Colovai Al, Ciubotariu R, Liu Z, Cortesini R, Suciu-Foca N: CD8(+)CD28(−) T suppressor cells represent a distinct subset in a heterogeneous population. Transplant Proc. 2001; 33:104-107.

15. Dhodapkar M V, Steinman R M, Krasovsky J, Munz C, Bhardwaj N: Antigen specific inhibition of effector T cell function in humans after injection of immature dendritic cells. J Exp Med. 2001; 193:233-238

16. Dhodapkar M V, Steinman R M, Sapp M, Desai H, Fossella C, Krasovsky J, Donahoe S M, Dunbar P R, Cerundolo V, Nixon D F, Bhardwaj N: Rapid generation of broad T-cell immunity in humans after a single injection of mature dendritic cells. J. Clin. Invest. 1999; 104:173-180

17. Dhodapkar, M., and N. Bhardwaj. 2000. Active immunization of humans with dendritic cells. Journal of Clinical Immunology 20:167-174.

18. Dhodapkar, M. V., J. Krasovsky, R. Steinman, and N. Bhardwaj. 2000. Mature dendritic cells boost functionally superior T cell in humans without foreign helper epitopes. J Clin. Invest. 107:R9-R14.

19.Dhodapkar, M. V., R. M. Steinman, M. Sapp, H. Desai, C. Fossella, J. Krasovsky, S. M. Donahoe, P. R. Dunbar, V. Cerundolo, D. F. Nixon, and N. Bhardwaj. 1999. Rapid generation of broad CD8+T-cell immunity in humans after a single injection of mature dendritic cells. J. Clin. Invest. 104:173-180.

20. Dieckmann D, Plottner H, Berchtold S, Berger T, Schuler G: Ex vivo isolation and characterization of CD4(+)CD25(+) T cells with regulatory properties from human blood. J Exp Med. 2001; 193:1303-1310.

21. Filaci G, Bacilieri S, Fravega M, Monetti M, Contini P, Ghio M, Setti M, Puppo F, Indiveri F: Impairment of CD8+T suppressor cell function in patients with active systemic lupus erythematosus. J Immunol. 2001; 166:6452-6457.

22. Gaur A, Ruberti G, Haspel R, Mayer J P, Fathman C G: Requirement for CD8+ cells in T cell receptor peptide-induced clonal unresponsiveness. Science. 1993; 259:91-94.

23. Groux H, O'Garra A, Bigler M, Rouleau M, Antonenko S, de Vries J E, Roncarolo M G: A CD4+T-cell subset inhibits antigen-specific T-cell responses and prevents colitis. Nature. 1997; 389:737-742.

24. Hart, D. N. J. and J. L. McKenzie. 1990. Interstitial dendritic cells. Intl. Rev. Immunol. 6,128.

25. Huang, F. P., N. Platt, M. Wykes, J. R. Major, T. J. Powell, C. D. Jenkins, and G. G. MacPherson. 2000. A discrete subpopulation of dendritic cells transports apoptotic intestinal epithelial cells to T cell areas of mesenteric lymph nodes. J Exp Med 191:435-444.

26. Inaba K, Turley S, Iyoda T, Yamaide F, Shimoyama S, Reis e Sousa C, Germain R N, Mellman I, Steinman R M: The formation of immunogenic major histocompatibility complex class II-peptide ligands in lysosomal compartments of dendritic cells is regulated by inflammatory stimuli. J Exp Med. 2000; 191:927-936

27.Inaba, K., J. P. Metlay, M. T. Crowley, and R. M. Steinman. 1990a. Dendritic cells pulsed with protein antigens in vitro can prime antigen-specific, MHC-restricted T cells in situ. J. Exp. Med. 172,631.

28. Inaba, K., J. P. Metlay, M. T. Crowley, M. Witmer-Pack, and R. M. Steinman. 1990b. Dendritic cells as antigen presenting cells in vivo. Intl. Rev. ImmunoL 6, 197.

29. Inaba, K., Turley, S., Iyoda, T., Yamaide, F., Shimoyama, S., Reis, E., Sousa, C., Germain, R. N., Mellman, I., and R. M. Steinman. 2000. The formation of immunogenic major histocompatibility complex class II-peptide ligands in lysosomal compartments of dendritic cells is regulated by inflammatory stimuli. J Exp Med Mar 20:191 (6) :927-36.

30.Jonuleit H, Schmitt E, Schuler G, Knop J, A. E: Induction of human IL-10 producing, non-proliferating CD4+T cells with regulatory properties by repetitive stimulation with allogeneic immature dendritic cells. J Exp Med. 2000; 192:1213-1222

31.Jonuleit, H., E. Schmitt, G. Schuler, J. Knop, and A. H. Enk. 2000. Introduction of Interleukin 10-producing, Nonproliferating CD4+T Cells with Regulatory Property by Repetitive Human Dendritic Cells. J Exp Med. 192:1213-1222.

32. Koh D-R, Fung-Leung W-P, Ho A, Gray D, Acha-Orbea H, Mak T-W: Less mortality but more relapses in experimental allergic encephalomyelitis in CD8 -/- mice. Science. 1992; 256:1210-1213

33. Koide J, Engleman E G: Differences in surface phenotype and mechanism of action between alloantigen-specific CD8+ cytotoxic and suppressor T cell clones. J Immunol. 1990; 144:32-40.

34. Labeur, M. S., B. Roters, B. Pers, A. Mehling, T. A. Luger, T. Schwarz, and S.Trabbe. 1999. Generation of tumor immunity by bone marrow-derived dendritic cells correlates with dendritic cell maturation stage. J Immunol Jan 1;162(1):168-75.

35. Lee, P. P., C. Yee, P. A. Savage, L. Fong, D. Brockstedt, J. S. Weber, D. Johnson, S. Swetter, J. Thompson, P. D. Greenberg, M. Roederer, and M. M. Davis. 1999. Characterization of circulating T cells specific for tumor-associated antigens in melanoma patients. Nat Med 5:677-685.

36. Levings M K, Sangregorio R, Roncarolo M G: Human CD25(+)CD4(+) T regulatory cells suppress naive and memory T cell proliferation and can be expanded in vitro without loss of function. J Exp Med. 2001; 193:1295-1302.

37. Lutz, M. B., R. M. Suri, M. Niimi, A. L. Ogilvie, N. A. Kukutsch, W. Rossner, G. Schuler, and J. M. Austyn. 2000. Immature dendritic cells generated with low doses of GM-CSF in the absence of IL-4 are maturation resistant and prolong allograft survival in vivo. Eur J Imminol Jul;30(7): 1813-22.

38.Macatonia, S. E., T. M. Doherty, S. C. Knight, and A. O'Garra. 1993. Differential effect of IL-10 on dendritic cell-induced T cell proliferation and IFN-gamma production. J Immunol 150:3755-3765.

39. Mellman I, Steinman R M: Dendritic cells: specialized and regulated antigen processing machines. Cell. 2001; 106:255-258.

40. Moore, K. W., A. O'Garra, R. Waal Malefyt, P. Vieira, T. R. Mossman. 1993. Interleukin 10. Ann Rev Immunol 11:165-190.

41. Panelli, M. C., J. Wunderlich, J. Jeffries, E. Wang, A. Mixon, S. A. Rosenberg, and F. M. Marincola. 2000. Phase 1 study in patients with metastatic melanoma of immunication with dendritic cells presenting epitopes derived from the melanoma-associated antigens MART-1 and gp100. J Immunother Jul-Aug;23 (4) :487-98.

42. Porgador, A. and E. Gilboa. 1995. Bone marrow-generated dendritic cells pulsed with a class I-restricted peptide are potent inducers of cytotoxic T lymphocytes. J Exp. Med.l 182, 255.

43. Roncarolo M G, Levings M K: The role of different subsets of T regulatory cells in controlling autoimmunity. Curr Opin Immunol. 2000; 12:676-683.

44. Sakaguchi S, Sakaguchi N, Asano M, Itoh M, Toda M: Immunologic self-tolerance maintained by activated T cells expressing IL-2 receptor alpha-chains (CD25). Breakdown of a single mechanism of self-tolerance causes various autoimmune diseases. J Immunol. 1995; 155:1151-1164.

45. Sakaguchi S: Regulatory T cells: key controllers of immunologic self-tolerance. Cell. 2000; 101:455-458

46. Sakaguchi, S. 2000. Regulatory T cells: key controllers of immunologic self-tolerance. Cell 101:455-458.

47. Shevach E M: Certified professionals: CD4(+)CD25(+) suppressor T cells. J Exp Med. 2001; 193:F41-46.

48. Steinman, R. M. 1991. The dendritic cell system and its role in immunogenicity. Annu. Rev. Immunol. 9, 271.

49.Steinman, R. M., S. Turley, I. Mellman, and K. Inaba. 2000. The induction of tolerance by dendritic cells that have captured apoptotic cells. J Exp Med 191:411-416.

50.Sutmuller R P, van Duivenvoorde L M, van Elsas A, Schumacher T N, Wildenberg M E, Allison J P, Toes R E, Offringa R, Melief C J: Synergism of cytotoxic T lymphocyte-associated antigen 4 blockade and depletion of CD25(+) regulatory T cells in antitumor therapy reveals alternative pathways for suppression of autoreactive cytotoxic T lymphocyte responses. J Exp Med. 2001; 194:823-832.

51.Taylor P A, Noelle R J, Blazar B R: CD4(+)CD25(+) immune regulatory cells are required for induction of tolerance to alloantigen via costimulatory blockade. J Exp Med. 2001; 193:1311-1318.

52.Thomson, A. W., and L. Lu. 1999. Are dendritic cells the key to liver transplant tolerance? Immunology Today 20:27-32.

53.Thurner, B., I. Haendle, C. Roder, D. Dieckmann, P. Keikavoussi, H. Jonuleit, A. Bender, C. Maczek, D. Schreiner, P. von den Driesch, E. B. Brocker, R. M. Steinman, A. Enk, E. Kampgen, and G. Schuler. 1999. Vaccination with mage-3A1 peptide-pulsed mature, monocyte-derived dendritic cells expands specific cytotoxic T cells and induces regression of some metastases in advanced stage IV melanoma. J Exp Med 190: 1669-1678.

54.VanBuskirk A M, Burlingham W J, Jankowska-Gan E, Chin T, Kusaka S, Geissler F, Pelletier R P, Orosz C G: Human allograft acceptance is associated with immune regulation. J Clin Invest. 2000; 106:145-155.

55.Waldmann H, Cobbold S: Regulating the immune response to transplants. a role for CD4+ regulatory cells? Immunity. 2001; 14:399-406.

56.Zajac, A. J., J. N. Blattman, K. Murali-Krishna, D. J. Sourdive, M. Suresh, J. D. Altman, and R. Ahmed. 1998. Viral immune evasion due to persistence of activated T cells without effector function. J Exp Med 188:2205-2213.

We claim:

1. A method of silencing or suppressing a pre-existing immune response to an antigen in a mammal, wherein said immune response is characterized by the presence of CD8+T cells which are specific for said antigen, and wherein said method comprises administering to said mammal a sufficient amount of immature dendritic cells which have been contacted with said antigen to suppress or silence said immune response.

2. The method according to claim 1 wherein the immature dendritic cells are derived from blood or bone marrow.

3. The method according to claim 1 wherein the mammal is human.

4. The method according to claim 1 wherein the antigen is insulin, glutamic acid decarboxylase (GAD), or islet associated autoantigen.

5. The method according to claim 1 wherein the antigen is myelin basic protein and/or proteolipid protein.

6. The method according to claim 1 wherein the antigen is acetylcholine receptor.

7. The method according to claim 1 wherein the antigens are lupus antigens selected from the group consisting of nuclear proteins, ribosomal proteins, nucleic acid protein complexes, and histones.

8. The method according to claim 1 wherein the antigens are autoantigen derived from stem cells or whole cell preparations from insulinoma, thymic tissue, or B lymphoblastoid cell lines.

9. The method according to claim 1 wherein the pre-existing immune response is an autoimmune disease selected from the group comprising juvenile diabetes, multiple sclerosis, myasthenia gravis, psoriasis, lupus, and atopic dermatitis.

10. The method according to claim 1 wherein the immature dendritic cells are contacted with antigen in vivo.

11. The method according to claim 10 wherein the immature dendritic cells are maintained in an immature state by administering to the immature dendritic cells an IL-10 gene expressing vector.

12. A method for silencing or suppressing the function of pre-existing CD8+T cells which are specific for an antigen in a mammal comprising:
(a) contacting immature dendritic cells with said antigen in vitro; and
(b) administering the immature dendritic cells to a mammal in an amount sufficient to silence or suppress said pre-existing antigen specific CD8+T cell function.

13. The method according to claim 12, wherein the tissue source is human.

14. The method according to claim 12, wherein the tissue source is blood or bone marrow.

15. The method according to claim 12, wherein the dendritic cells are contacted with a cytokine selected from the group consisting of GM-CSF, IL-4 or IL-13.

16. The method according to claim 12, further comprising administering the immature dendritic cells to a mammal in a pharmaceutically acceptable carrier.

17. The method according to claim 16, wherein between 1×10⁶ and 10×10⁶ immature dendritic cells are administered to a mammal per dose.

18. The method according to claim 1, wherein the immature dendritic cells are administered intravenously, subcutaneously, or intramuscularly.

19. A pharmaceutical composition comprising immature dendritic cells prepared according to claim 1 and a pharmaceutically acceptable carrier.

20. A pharmaceutical composition comprising the immature dendritic cells prepared according to claim 1 and a cytokine and a pharmaceutically acceptable carrier.

21. A kit for inhibiting the function of pre-existing antigen specific T cells, which kit comprises immature dendritic cells which have been contacted with said antigen.

22. A kit for maintaining immature dendritic cells in an immature state, which kit comprises the immature dendritic cells according to claim 21 and at least one vector comprising a gene encoding TGF-beta and/or IL-10.

23. A kit for inhibiting the function of pre-existing antigen specific T cells, which kit comprises immature dendritic cells and said antigen.

24. The method according to claim 1, wherein administration of the immature dendritic cells stimulates induction of antigen specific IL-10 producing CD8+T cells.

25. The method according to claim 1, wherein antigens are targeted in vivo to immature DCs resident in tissues or elicited after contact with cytokines such as FLT-3 ligand or G-CSF.

26. The method according to claim 1, wherein the immature dendritic cells are modified to prevent their maturation in vivo after injection into a mammal.

27. The method according to claim 1 wherein antigen specific regulatory CD8+T cells are generated in vivo for active immunotherapy.

28. A method for generating antigen specific regulatory CD8+T cells in vitro for adoptive immunotherapy, wherein said method comprises contacting T cells in vitro with immature dendritic cells containing an antigen for a time sufficient to generate antigen specific regulatory CD8+T cells, and administering said regulatory CD8+T cells to a mammal in an amount sufficient to suppress an immune response.
